(19) United States
(12) Patent Application Publication
(10) Pub. No.: US 2013/0171032 A1
(43) Pub. Date: Jul. 4, 2013

(54) ELECTRONIC INCENSE STICK HAVING HIGH LIGHT USAGE EFFICIENCY

(71) Applicant: PO-CHOU CHEN, Tu-Cheng (TW)

(72) Inventor: PO-CHOU CHEN, Tu-Cheng (TW)

(73) Assignee: HON HAI PRECISION INDUSTRY CO., LTD., Tu-Cheng (TW)

(21) Appl. No.: 13/491,579

(22) Filed: Jun. 7, 2012

(30) Foreign Application Priority Data
Jan. 2, 2012 (TW) 101100098

(51) Int. Cl. A61L 9/03

(52) U.S. Cl. 422/126

(57) ABSTRACT

An electronic incense stick includes a transparent tube, a first non-transparent coating, a second non-transparent coating, and a laser diode. The tube includes an opened end and a bulb portion formed at an opposite end. The first non-transparent coating is formed on a major part of an outer surface of the tube adjacent to the bulb portion. The second non-transparent coating is formed on a part of the outer surface that is not covered by the first non-transparent coating. The laser diode is positioned at the opened end and configured for emitting a laser light beam which transmits to the bulb portion along a central axis of the tube.

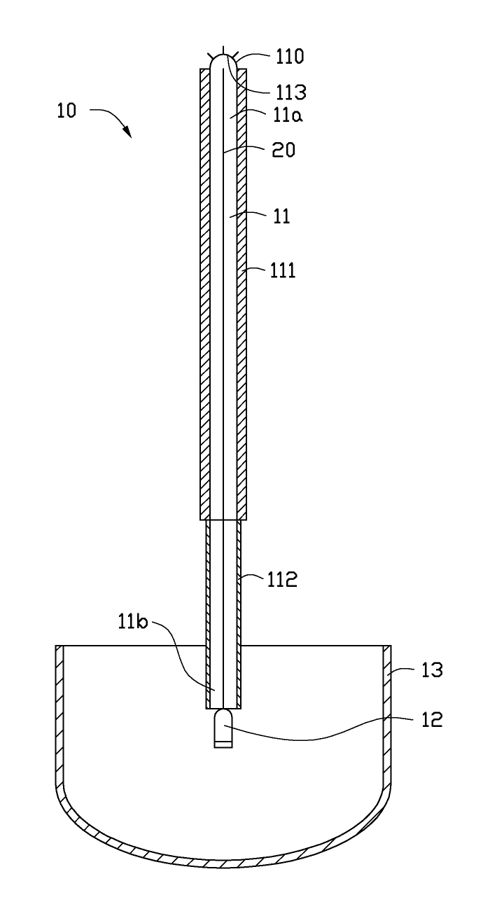

### BACKGROUND

1. Technical Field

The present disclosure relates to electronic incense sticks, and particularly, to an electronic incense stick with a high light usage efficiency.

2. Description of Related Art

Electronic incense sticks typically includes a transparent tube having an opened end and a bulb portion at an opposite end of the tube, non-transparent coatings on an outer surface of the tube to simulate an incense coating and a bone part of traditional incense sticks, and a light emitting diode (LED) positioned at the opened end. The LED emits light, which is directed to the bulb portion via multiple internal reflections on an inner surface of the tube. Thus, the electronic incense stick appears burning-like. However, light is unavoidably lost at the internal reflections, thus decreasing light usage efficiency.

Therefore, it is desirable to provide an electronic incense stick, which can overcome the above-mentioned shortcomings.

### BRIEF DESCRIPTION OF THE DRAWING

The FIGURE is a cross-sectional schematic view of an electronic incense stick, according to an embodiment.

### DETAILED DESCRIPTION

Embodiments of the disclosure will be described in detail, with reference to the accompanying drawing.

Referring to the FIGURE, an electronic incense stick 10, according to an embodiment, includes a transparent tube 11, a first non-transparent coating 111, a second non-transparent coating 112, and a laser diode 12.

The tube 11 having an opened end 11b and a bulb portion 110 at an opposite end 11a. The bulb portion 110 has a diffusion structure 113 for diffusing light. In this embodiment, the inner surface of the bulb portion 110 is roughened and serves as the diffusion structure 113.

The first non-transparent coating 111 is formed on a major part of an outer surface of the tube 11, adjacent to the bulb portion 110 to simulate incense coating of traditional incense sticks. Accordingly, the first non-transparent coating 111 may have a yellow or red color.

The second non-transparent coating 112 is formed on the outer surface of the tube 11 which is uncovered by the first non-transparent coating 111 to simulate the bone of the traditional incense sticks. Accordingly, the second non-transparent coating 112 has a wood-like color.

The laser diode 12 is positioned at the opened end 11b and configured for emitting a highly collimated laser beam 20 into the tube 11, which transmits to the bulb portion 110 along a central axis of the tube 11. Then, the light beam 20 is diffused by the diffusion structure 113 to light up the whole bulb portion 110. As such, the electronic incense stick 10 appears burning-like. The light usage efficiency is enhanced as the laser beam 20 can arrive at the bulb portion 110 without reflection and thus retains most if not all of the light.

In use, the electronic incense stick 12 can be stuck into a burner 13.

Particular embodiments are shown here and described by way of illustration only. The principles and the features of the present disclosure may be employed in various and numerous embodiments thereof without departing from the scope of the disclosure as claimed. The above-described embodiments illustrate the scope of the disclosure but do not restrict the scope of the disclosure.

1. An electronic incense stick, comprising:
a transparent tube comprising an opened end and a bulb portion formed at an opposite end;
a first non-transparent coating positioned on a major part of an outer surface of the tube adjacent to the bulb portion;
a second non-transparent coating positioned on a part of the outer surface that is not covered by the first non-transparent coating; and
a laser diode positioned at the opened end and configured for emitting a laser light beam which transmits to the bulb portion along a central axis of the tube;
wherein the first non-transparent coating is thicker than the second non-transparent coating.

2. The electronic incense stick of claim 1, wherein the bulb portion comprises a diffusion structure for diffusing the laser light beam.

3. The electronic incense stick of claim 2, wherein an inner surface of the bulb portion is roughened and serves as the diffusion structure.

4. (canceled)

5. The electronic incense stick of claim 1, wherein the first non-transparent coating has a yellow or red color.

6-8. (canceled)

9. The electronic incense stick of claim 1, wherein the first non-transparent coating and the second non-transparent coating cooperatively cover the entire outer surface of the tube.
